(19) United States
(12) Patent Application Publication
(10) Pub. No.: US 2026/0182912 A1
(43) Pub. Date: Jul. 2, 2026

(54) SYSTEM USING HEART RATE VARIABILITY FEATURES FOR PREDICTION OF MEDICAL PROCEDURE EFFICACY

(71) Applicant: Medtronic, Inc., Minneapolis, MN (US)

(72) Inventors: Francisco Javier Saiz Vivo, L' Eliana (ES); Mirko De Melis, Maastricht (NL); Luca Mainardi, Fagnano Olona (IT); Valentina D.A. Corino, Milano (IT)

(21) Appl. No.: 18/868,027

(22) PCT Filed: May 22, 2023
(86) PCT No.: PCT/US2023/023131

§ 371 Date: Nov. 21, 2024

Related U.S. Application Data
(60) Provisional application No. 63/365,188, filed on May 23, 2022.

(51) Int. Cl. A61B 5/00; A61B 5/024; A61B 5/352; A61B 5/36

(52) U.S. Cl. CPC A61B 5/4848; A61B 5/02405; A61B 5/352; A61B 5/36; A61B 5/742

(57) ABSTRACT

A method including collecting, by a computing system, heart rate data of a patient from a medical device of the patient; determining, by the computing system, one or more heart rate variability features based on the heart rate data; applying, by the computing system, a model to the heart rate variability features and one or more clinical features of the patient; predicting, by the computing system, an effect of a medical procedure on the patient based on the application of the model to the heart rate variability features and the one or more clinical features; and outputting, by the computing system, the predicted effect of the medical procedure to a display device.

This application claims the benefit of U.S. Provisional Patent Application Ser. No. 63/365,188, filed May 23, 2022, and entitled “SYSTEMS USING HEART RATE VARIABILITY FEATURES FOR PREDICTION OF MEDICAL PROCEDURE EFFICACY,” the entire contents of which is incorporated herein by reference.

### TECHNICAL FIELD

This disclosure relates to medical device systems and, more particularly, to medical device systems for monitoring efficacy of medical treatments.

### BACKGROUND

In some situations, medical professionals may perform various medical procedures to cardiac-related tissues of a patient to treat various medical conditions. The various medical procedures may or may not be successful in addressing the various medical conditions.

### SUMMARY

The devices, systems, and techniques of this disclosure generally relate to prediction of effects of therapies on cardiac tissues of a patient. In some examples, a computing system in accordance with this disclosure may predict the efficacy and/or effects of one or more medical procedures directed at cardiac tissue of the patient based on heart rate data. In some examples, the computing system may predict the effects of a medical procedure based on application of models to heart rate variability features and clinical features of the patient. In some examples, the computing system may output the predicted effects of the medical procedure (e.g., to a medical professional). The medical procedure may be catheter ablation for atrial fibrillation (AF).

The devices, systems, and techniques of this disclosure may provide one or more technical improvements over other medical procedure efficacy prediction techniques. In some examples, the disclosure describes techniques that improve predicative accuracy of the efficacy of a medical procedure. The disclosure may improve the predicative accuracy by using combinations of heart rate variability feature(s) and clinical feature(s) that are demonstrated to be predictive of the efficacy of the medical procedure. In some examples, the disclosure describes techniques that improve the predicative accuracy procedure by using weighted combination of a plurality of classification models to improve the overall accuracy of the techniques. Moreover, the model(s) used to predict the efficacy of the procedure may be machine learning models trained on numerous (thousands or millions) of instances of training data to provide highly accurate predictions exceeding conventional techniques for estimating procedure efficacy. Additionally, in some examples, the heart rate variability feature(s) may be determined based on cardiac signals sensed continuously sensed (e.g., autonomously on a triggered or periodic basis) by an insertable cardiac monitor (ICM) or other implantable medical device (IMD), which may provide a much more complete picture of the condition of the patient than could be determined by a clinician using conventional clinical evaluation techniques. AF episodes may occur infrequently and/or unpredictably, but an IMD continuously sensing cardiac signals may sense all AF episodes that the patient experiences.

In an example, the disclosure describes a method including collecting, by a computing system, heart rate data of a patient from medical device of the patient; determining, by the computing system, one or more heart rate variability features based on the heart rate data; applying, by the computing system, a model to the heart rate variability features and one or more clinical features of the patient; predicting, by the computing system, an effect of a medical procedure on the patient based on the application of the model to the heart rate variability features and the one or more clinical features; and outputting, by the computing system, the predicted effect of the medical procedure to a display device.

In some examples, the disclosure describes a computing system including memory configured to store heart rate data; a display device; and processing circuitry configured to: collect heart rate data of the patient from a medical device of the patient; determine one or more heart rate variability features based on the heart rate data; apply a model to the heart rate variability features and one or more clinical features of the patient; predict an effect of a medical procedure on the patient based on the application of the model to the heart rate variability features and the one or more clinical features; and output the predicted effect of the medical procedure to the display device.

In some examples, the disclosure describes computer readable storage medium including instructions that, when executed, cause processing circuitry within a device to perform a method including collecting, by a computing system, heart rate data of a patient from medical device of the patient; determining, by the computing system, one or more heart rate variability features based on the heart rate data; applying, by the computing system, a model to the heart rate variability features and one or more clinical features of the patient; predicting, by the computing system, an effect of a medical procedure on the patient based on the application of the model to the heart rate variability features and the one or more clinical features; and outputting, by the computing system, the predicted effect of the medical procedure to a display device.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram of a medical device system for predicting effects of a medical procedure on a patient.

FIG. 2 is a block diagram illustrating an example configuration of an implantable medical device (IMD) of the system of FIG. 1.

FIG. 3 is a block diagram illustrating an example external device of the system of FIG. 1.

FIG. 4 is a block diagram illustrating an example health monitoring system (HMS) of the system of FIG. 1.

FIG. 5 is a conceptual diagram illustrating an example set of heart rate data recorded by an IMD of the system of FIG. 1.

FIG. 6 is a conceptual diagram illustrating an example neural network configured to predict the effects of the medical procedure.

FIG. 7 is a conceptual diagram illustrating an example process of inputting data into an example model for the prediction of the effects of the medical procedure.

FIG. 8 is a block diagram illustrating an example process of training an example model for the prediction of the effects of the medical procedure.

FIG. 9 is a flowchart illustrating an example process of predicting effects of a medical procedure.

FIG. 10 is a flowchart illustrating an example process of generating a model for the prediction of the effects of the medical procedure.

### DETAILED DESCRIPTION

Medical devices, systems, and techniques of this disclosure relates to prediction of effects of therapies on cardiac tissues of a patient. A medical professional may perform one or more medical procedures to cardiac tissue of a patient to treat one or more medical conditions experienced by the patient. The medical professional may perform the one or more medical procedures on the patient to treat atrial fibrillation (AF). In some examples, the one or more medical procedures includes cardiac ablation techniques such as, but is not limited to, catheter ablation or pulmonary vein isolation (PVI). In some examples, the medical professional selects cardiac ablation over other treatment procedures (e.g., an antiarrhythmic drug therapy) for patients that do not respond well to the other treatment procedures or vice versa.

The medical professional may select a medical procedure from a plurality of available medical procedures based on the symptoms of the patient. For example, the patient may be highly symptomatic. In some examples, with respect to patients experiencing AF, the patient may experience paroxysmal AF (PAF) or non-paroxysmal AF (NPAF). In some examples, the efficacy, such as short-term efficacy and/or long-term (e.g., greater than 12 months) efficacy, of the medical procedure may be limited and there may be additional risks to the health of the patient as a result of undergoing the procedure. Thus, a medical professional may desire to predict the effect of the medical procedure on the health of the patient and/or the efficacy of the medical procedure on the medical condition of the patient prior to performance of the medical procedure on the patient.

Medical professionals may use existing scoring systems to predict the outcome of a medical procedure on cardiac tissue of the patient. The scoring system may include risk predictors including, but are not limited to, thromboembolic risk predictors (e.g., CHADS₂, CHA₂DS₂-VASc, or the like), the APPLE score, the SUCCESS score, the MB-LATER score, or the like. However, the existing scoring systems rely on monitoring techniques (e.g., 24-hour Holter monitoring) which may lack adequate sensitivity and detection of medical conditions (e.g., AF recurrences) under certain conditions. For example, the monitoring techniques exhibit inadequate detection rates for subclinical AF recurrences.

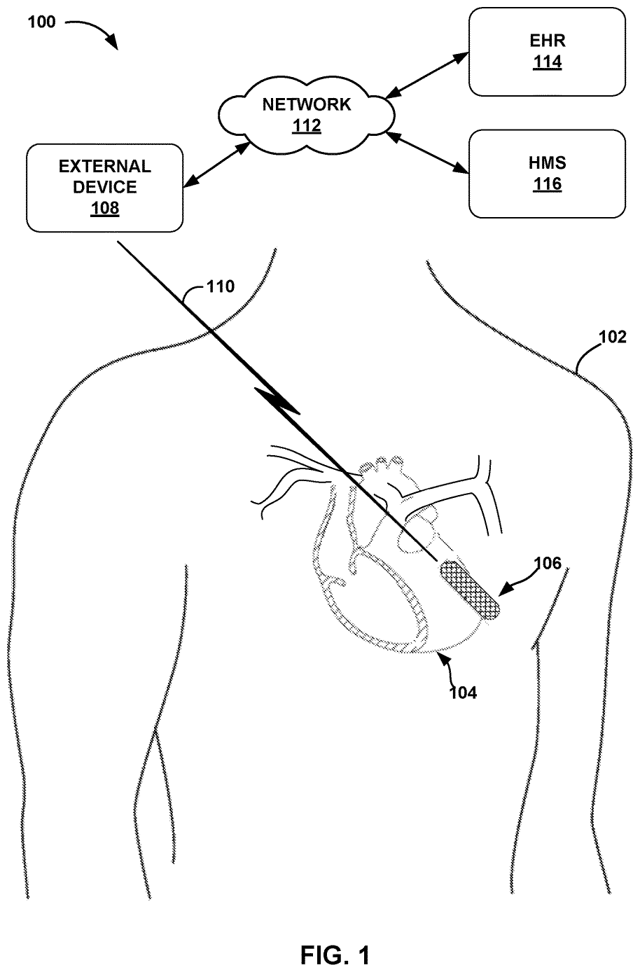

FIG. 1 is a conceptual diagram of a medical device system 100 for predicting effects of a medical procedure on a patient 102. Medical device system 100 may include an implantable medical device (IMD) 106, an external device 108, a network 112, an electronic health record (EHR) system 114, and a health monitoring system (HMS) 116. While the discussion below and elsewhere in this disclosure describes an implantable medical device (e.g., IMD 106), other example medical device systems may include an external medical device that provides functionality that is the same or substantially similar to that ascribed to IMD 106 herein.

IMD 106 may be configured to detect and record heart rate data from heart 104 of patient 102. In some examples, IMD 106 detects and records heart rate data by detecting and recording depolarization of one or more chambers (e.g., left ventricle (LV), right ventricle (RV), left atrium (LA), or right atrium (RA)) of heart 104. IMD 106 may detect and record heart rate of patient 102 by detecting and recording QRS complexes corresponding to ventricular depolarization of heart 104. IMD 106 may determine, based on the recorded QRS complexes, R-R intervals for the ventricular depolarization of heart 104. Each R-R interval may represent a time between R-waves of adjacent QRS complexes.

External device 108 may be one or more computing devices, one or more computing systems, and/or a cloud computing environment. IMD 106 may be configured to communicate with and transmit recorded heart rate data 110 to external device 108. IMD 106 and external device 108 may communicate wirelessly or via a wired communication. External device 108 may further communicate with a cloud network 112 and communicate information (e.g., heart rate data 110) between one or more EHR systems 114 and one or more HMS 116 via network 112. In some examples, external device 108 determines heart rate variability (HRV) features and clinical features of patient 102 based on information from IMD 106, EHR system 114, and/or HMS 116.

External device 108 may determine HRV features based at in part on heart rate data 110. HRV features may act as a predictor for recurrence of medical conditions including, but is not limited to, atrial fibrillation (AF). HRV features may include one or more of, but is not limited to: an average of the recorded R-R intervals (hereinafter referred to as “Mean value”), percentage of the interval differences of successive R-R intervals greater than a time threshold (pNNX), mean square differences of successive R-R intervals (RMSSD), standard deviation of the R-R intervals (SDNN), triangular interpolation of interval histogram (TINN), triangular index (TRI), approximate entropy (ApEn), sample entropy (SampEn), Geometric descriptors of a Poincare plot of heart rate data 110 (SD1, SD2, SD1:SD2 ratio, or the like), scaling exponent of short-term fluctuations in heart rate data 110 (DFA α1), or scaling exponent of long-term fluctuations in heart rate data 110 (DFA α2). pNNX may include a percentage of interval differences of successive R-R intervals greater than 50 milliseconds (ms) (pNN50) or greater than 20 ms (pNN20). TRI may describe an integral of a density distribution of heart rate data 110. ApEn and SampEn may represent a complexity of heart rate data 110 (e.g., a complexity of recorded R-R intervals).

External device 108 may determine clinical features of patient 102 based at least in part on information received from one or more EHR systems 114 and/or one or more HMS 116. The clinical features may include, but are not limited to, age of patient 102, presence of any illnesses in patient 102, or monitoring time of patient 102 prior to a medical procedure. The illnesses may include any illnesses that may affect the cardiac health of patient 102 including, but is not limited to: PAF, hypertension, diabetes, coronary artery disease, lesions, or stroke. In some examples, external device 108 receives, for each of the clinical features, a baseline characteristic from network 112. The baseline characteristics may include, for each of the clinical features, an average value for patients that experienced a recurrence of the medical condition and an average value for patients that experienced no recurrence of the medical condition.

External device 108 may determine and/or apply one or more models to one or more HRV features and the one or more clinical features to predict an effect and/or efficacy of the medical procedure on patient 102. The determination and application of the one or more models are described in greater detail below. In other examples, network 112 and/or one or more other computing systems, computing devices, and/or cloud computing environments may be configured to determine the one or more models and/or apply the one or more models. In some examples, external device 108 and/or network 112 is further configured to output the predicted effects to a display device. The display device may be incorporated into external device 108 or may be incorporated into another computing device, or computing system in communication with external device 108 and/or network 112.

External device 108 may be configured to communicate with a variety of other computing devices and/or computing systems via network 112. External device 108 and/or network 112 may comprise, or may be implemented by, the Medtronic CarelinkTMNetwork. External device 108 may include one or more of a desktop, laptop, tablet computer, smartwatch, personal computing device, or the like. External device 108 may wirelessly communicate with IMD 106 and/or network 112 according to one or more wireless communications protocols (e.g., according to the Bluetooth® or Bluetooth® Low Energy (BLE) protocols).

Network 112 may facilitate connection between external device 108, one or more HMS 116, and one or more EHR system 114. HMS 116 is implemented on external device 108 and/or one or more other computing devices, one or more other computing systems, or a cloud computing environment. HMS 116 may retrieve data regarding patient 102 from one or more sources of EHR via network 112. In some examples, EHR is stored in EHR system 114. EHR system 114 may be implemented on external device 108 and/or one or more computing devices, one or more computing systems, or a cloud computing environment.

EHR data may include data regarding historical (e.g., baseline) physiological parameter values, previous health events and treatments, disease states, comorbidities, demographics, height, weight, and body mass index (BMI), as examples, of patients including patient 102. HMS 116 may use date from EHRs (e.g., from EHR system 114) to configure the one or more models implemented by medical device system 100 to predict effects of the medical procedure. In some examples, HMS 116 and/or EHR system 114 provide data from one or more EHRs to external device 108 for storage herein and use as part of the determination and/or application of the one or more models for predicting the effects of the medical procedure.

Network 112 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 112 may include one or more networks administered by service providers, and may thus form part of a large-scale public network infrastructure, e.g., the Internet. Network 112 may provide computing devices and systems, such as those illustrated in FIG. 1, access to the Internet, and may provide a communication framework that allows the computing devices and systems to communicate with one another. In some examples, network 112 includes a private network that provides a communication framework that allows the computing devices and computing systems illustrated in FIG. 1 to communicate with each other, but isolates some of the data flowing from devices external to the private network for security purposes. In some examples, the communications between the computing devices and computing systems illustrated in FIG. 1 are encrypted.

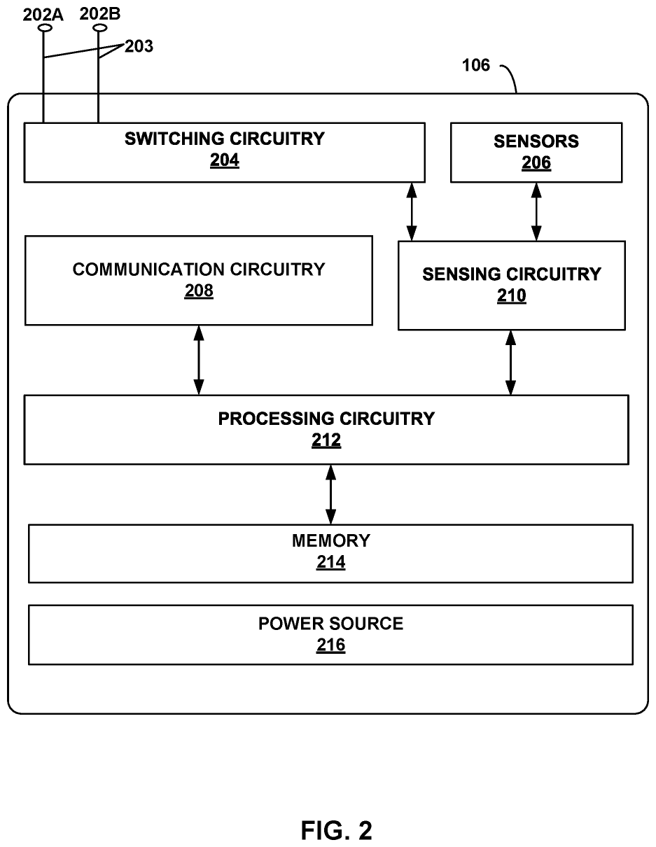

FIG. 2 is a block diagram illustrating an example configuration of an IMD 106 of the medical device system 100 of FIG. 1. IMD 106 may be an implanted cardiac device including, but is not limited to, an implantable cardiac monitor (ICM), such as the LINQ II insertable cardiac monitor, available from Medtronic, Inc., an implantable pulse generator (IPG), implantable cardioverter defibrillator (ICD), a cardiac resynchronization therapy (CRT) device, or the like. In the example shown in FIG. 2, IMD 106 includes switching circuitry 204, electrodes 202A-B, sensors 206, communication circuitry 208, sensing circuitry 210, processing circuitry 212, memory 214, and power source 216. The various circuitry may be, or include, programmable or fixed function circuitry configured to perform the functions attributed to respective circuitry.

Memory 214 may store computer-readable instructions that, when executed by processing circuitry 212, cause IMD 106 to perform various functions. Memory 214 may be a storage device or other non-transitory medium. Memory 214 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

In some examples, IMD 106 may include additional components (e.g., a signal generation circuitry for delivery of therapeutic signals, or the like). In some examples, where the functions of IMD 106 are performed by an external medical device, the components of IMD 106 may be disposed in one or more computing devices, one or more computing systems, and/or a cloud computing environment.

Electrodes 202A-B (collectively referred to as “Electrodes 202”) are electrically connected to chambers of heart 104. Electrodes 202 may be electrically connected to switching circuitry 204 of IMD 106 through electrical connectors 203. Each of electrodes 202 may be electrically connected to a difference chamber of heart 104. While the example illustrated in FIG. 2 includes two electrodes 202, other examples may include or three or more electrodes 202.

Switching circuitry 204 may selectively couple sensing circuitry 210 to selected combinations of electrodes 202, e.g., to sense the electrical activity of the atria and/or the ventricles of heart 104. Sensing circuitry 204 may include filters, amplifiers, analog-to-digital converters, or other circuitry configured to sense cardiac electrical signals via electrodes 202. In some examples, sensing circuitry 210 is configured to detect events, e.g., depolarizations, within the cardiac electrical signals, and provide indications thereof to processing circuitry 212. In this manner, processing circuitry 212 may determine heart rate data 110 based on the sensed cardiac electrical signals and may store the determined heart rate data 110 to memory 214.

Processing circuitry 212 may include any one or more of a microprocessor, a controller, a digital signal processor (DSP), an application-specific integrated circuit (ASIC), a field-programmable gate array (FPGA), discrete logic circuitry, or any other processing circuitry configured to provide the functions attributed to processing circuitry 212 herein and may be embodied as firmware, hardware, software, or any combination thereof.

Processing circuitry 212 may determine heart rate data 110 based on sensed cardiac electrical signals from sensing circuitry 204 and may store heart rate data 110 into memory 214. Processing circuitry 212 may represent heart rate data 110 as QRS complexes representing ventricular depolarization of the ventricles of heart 104. In some examples, processing circuitry 212 transmits the QRS complexes to external device 108 via communication circuitry 208.

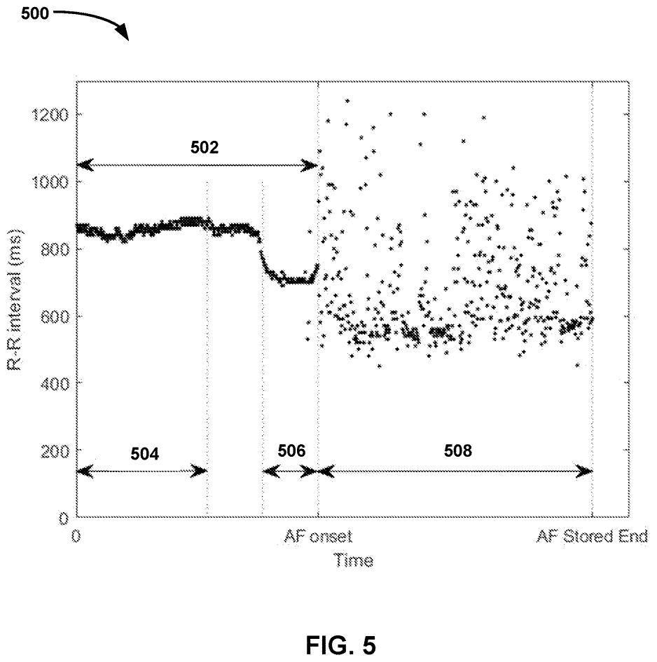

Processing circuitry 212 may determine the presence of AF based on the sensed cardiac signals. Processing circuitry 212 may apply one or more detection algorithms (e.g., TruRhythm™ available from Medtronic, Inc.) to the sensed cardiac signals to determine and record AF. In some examples, processing circuitry 212 may determine heart rate data 110 corresponding to AF episodes (e.g., an onset of the AF episode and a flashback of the AF episode, as illustrated in FIG. 5) and may store the determined heart rate data 110 in memory 214.

Sensors 206 may include one or more sensing elements that transduce patient physiological activity to an electrical signal to sense values of a respective patient parameter. Sensors 206 may include one or more accelerometers, optical sensors, chemical sensors, temperature sensors, pressure sensors, or any other types of sensors. Sensors 206 may output patient parameter values that may be used by processing circuitry 212 to determine heart rate data 110.

Communication circuitry 208 (alternatively referred to as “telemetry circuitry 312”) supports wireless communication between IMD 106 and external device 108. Processing circuitry 212 of IMD 106 may receive, from external device 108 and via communication circuitry 208, instructions to transmit heart rate data 110 to external device 108. In some examples, processing circuitry 212 automatically transmits heart rate data 110 to external device 108. Communication circuitry 208 may communicate with external device 108 via wired communication or by wireless communication techniques. Wireless communication techniques may include radiofrequency (RF) communication techniques, e.g., via an antenna (not shown). Communication circuitry 208 may transmit all of heart rate data 110 determined by processing circuitry 212. In some examples, communication circuitry 208 may transmit heart rate data 110 determined by processing circuitry 212 to correspond to AF episodes, e.g., as illustrated in FIG. 5.

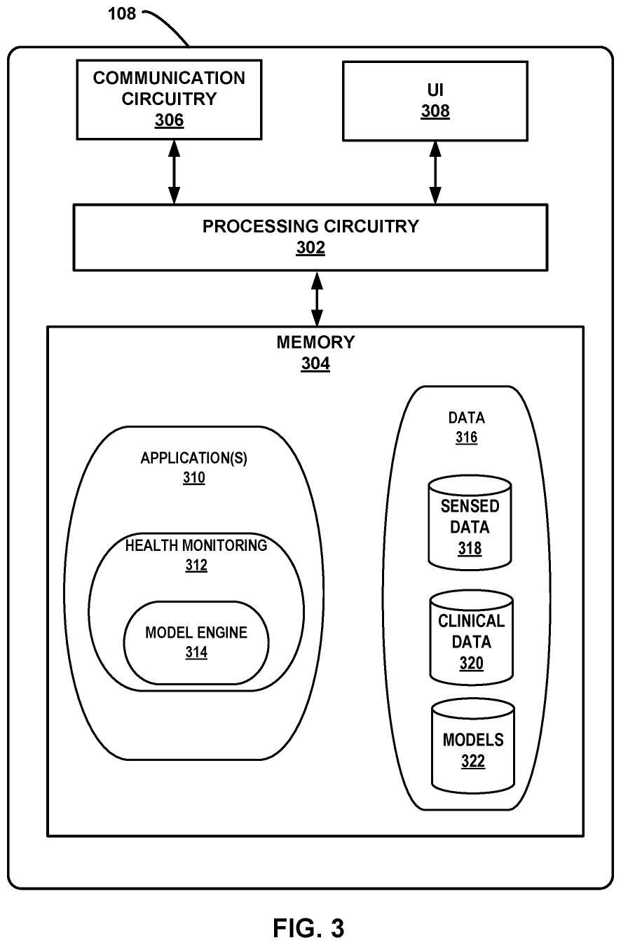

FIG. 3 is a block diagram illustrating an example external device 108 of the medical device system 100 of FIG. 1. As shown in FIG. 2, external device 108 includes processing circuitry 302, memory 304, communication circuitry 306, and user interface (UI) 308. Memory 304 may include one or more modules including application(s) module 310 and data module 312. Application(s) module 310 may include health monitoring module 312 which may include rules engine module 314. Data 316 stored in memory 304 may include sensed data 318, clinical data 320, and models 322. In some examples, data 312 may be separately stored in EHR system 114 and/or HMS 116.

Processing circuitry 302 may include fixed function circuitry and/or programmable processing circuitry. Processing circuitry 302 may include any one or more of a microprocessor, a controller, a GPU, a TPU, a digital signal processor (DSP), an ASIC, a FPGA, or equivalent discrete or analog logic circuitry. In some examples, processing circuitry 302 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 302 herein may be embodied as software, firmware, hardware, or any combination thereof. In some examples, memory 304 includes computer-readable instructions that, when executed by processing circuitry 302, cause external device 108 and processing circuitry 302 to perform various functions and/or processes attributed herein to external device 108, network 112, and/or processing circuitry 302. Memory 304 may include any volatile, non-volatile, magnetic, optical, or electrical media, such as a RAM, ROM, NVRAM, EEPROM, flash memory, or any other digital media.

Processing circuitry 302 may determine HRV features based on the received heart rate data 110, stored as sensed data 318. Heart rate data 110 may include time series of heart rate values associated with episodes or other significant time periods, determined by IMD 106 as described above. Processing circuitry 302 may determine the HRV features by executing instructions from memory 304 to perform mathematical algorithms corresponding to each of the HRV features. For example, processing circuitry 302 may determine the HRV feature of the Mean value by determining an average of the determined R-R intervals.

In some examples, processing circuitry 302 is configured to determine and/or apply one or more models for predicting the effects of a medical procedure on patient 102. In some examples, the determination and/or application of the models may be implemented by one or more other computing devices, computing systems, and/or cloud computing environments connected to network 112 and/or external device 108. Processing circuitry of the computing device(s), e.g., processing circuitry 302, may apply the one or more models to one or more HRV features and/or one or more clinical features stored in memory 304 and/or received by communication circuitry 306 from one or more EHR systems 114 and/or HMS 116.

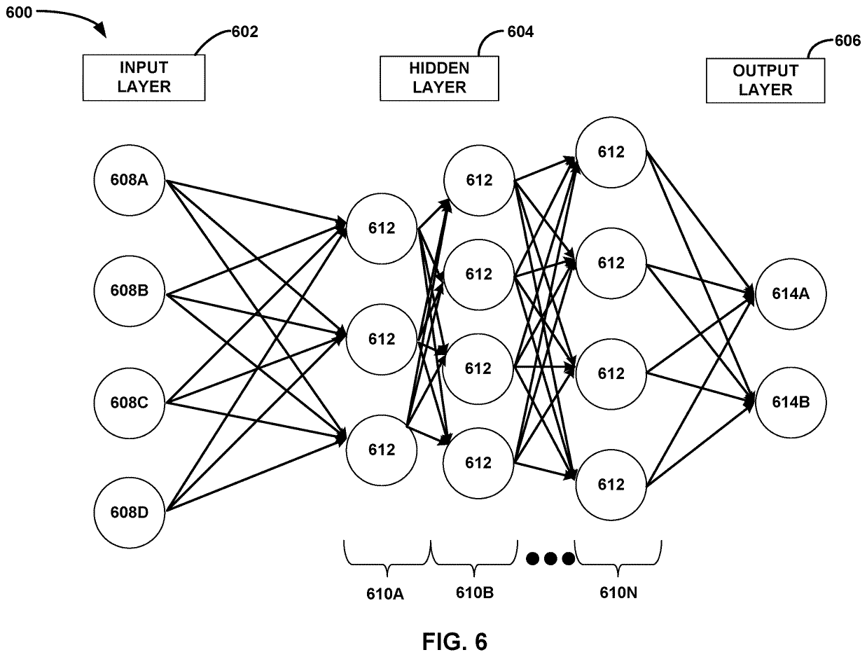

The one or more models may be configured for one or more features of patient 102. Processing circuitry 302 may determine portions of the one or more models through training using machine learning techniques (e.g., as described in greater detail in FIGS. 6-8). In some examples, processing circuitry 302 may determine one or more input values (e.g., HRV features, clinical features) for the one or more models by training using one or more machine learning techniques. The one or more models may include rule-based expert systems and/or trained ML models. In some examples, the one or more models may include a rules-based expert system and processing circuitry 302 may determine rules for the one or more rule-based expert system, e.g., through training using machine learning techniques. In some examples, the one or more models may include one or more trained ML models, and processing circuitry 302 may define and train the one or more trained ML models using machine learning techniques. In some examples, processing circuitry 302 may automatically define the entirety of a trained ML model through training using machine learning techniques. In some examples, the trained ML model may not include any individual rules. Example machine learning techniques may include, but are not limited to, supervised learning, and semi-supervised learning. In some examples, processing circuitry 302 may train the one or more models using one or more algorithms including, but are not limited to, Bayesian algorithms, Clustering algorithms, decision-tree algorithms, regularization algorithms, regression algorithms, instance-based algorithms, artificial neural network algorithms, deep learning algorithms, or dimensionality reduction algorithms. In some examples, processing circuitry 302 may determine a model by selecting input features (e.g., HRV features, clinical features) through training using machine learning techniques. During training using the ML models by inputting values of patient 102 for one or more features (e.g., HRV features, clinical features) and output a predicted effect of the medical procedure. In some examples, the predicted effect is the likelihood of recurrence of the medical condition, e.g., within 12 months after administration of the medical procedure.

Memory 304 is configured to store information within external device 108, e.g., for processing during operation of external device 108. Memory 304 may be described as a computer-readable storage medium. In some examples, memory 304 includes temporary memory or a volatile memory including, but is not limited, random access memories (RAM), dynamic random access memories (DRAM), static random access memories (SRAM), or other forms of volatile memories known in the art. Memory 304, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable memories (EEPROM). In some examples, memory 304 includes cloud-associated storage.

Communication circuitry 306 may facilitate communication between processing circuitry 302 of external device 108 and IMD 106, network 112, one or more EHR systems 114, HMS 116, and/or one or more other computing devices, computing systems, and/or cloud computing environments connected to network 112. In some examples processing circuitry 302 may transmit the trained ML model to one or more of HMS 116, network 112, or one or more other computing devices, computing systems, and/or cloud computing environments connected to network 112. Communication circuitry 306 may communicate with other devices and/or systems via wired and/or wireless communication techniques. Wireless communication techniques may include RF communication techniques, e.g., via an antenna (not shown). Communication circuitry 306 may include a radio transceiver configured for communication according to standards of protocols, such as 3G, 4G, 5G, WiFi (e.g., 802.11 or 802.15 ZigBee), Bluetooth®, or Bluetooth® Low Energy (BLE).

UI 308 may be configured to receive input, e.g., from patient 102 or another user. Examples of input are tactile, audio, kinetic, or optical input. UI 308 may include a mouse, a keyboard, voice responsive system, a camera, buttons, a control pad, a microphone, a presence-sensitive or touch-sensitive component (e.g., a screen), or any other device for detecting input from a user.

UI 308 may also be configured to generated output, e.g., to patient 102 or another user. Examples of output include tactile, haptic, audio, or visual output. UI 308 of external device 108 may include a presence-sensitive screen, a sound card, a video graphics adapter card, speakers, a cathode ray tube (CRT) monitor, a liquid crystal display (LCD), light emitting diodes (LEDs), or any other type of device for generating output to a user.

In some examples, as illustrated in FIG. 3, application(s) 310 may be executed in a user space in external device 108. As a part of the execution of application(s) 310, processing circuitry 302 may apply the one or more models to predict effects of a medical procedure on patient 102.

Application(s) 310 stored in memory 304 may include health monitoring module 312 (also referred to as “health monitoring layer 312”) which includes model engine module 314. Health monitoring module 312 may be responsive to receipt of a user request to predict effects of a particular medical procedure. Health monitoring module 312 may control performance of any of the operations in response to receiving the user request ascribed herein to external device 108, such as predicting effects of the medical procedure, and outputting the predicted effects to the user, e.g., via UI 308.

Model engine module 314 applies one or more models (e.g., trained ML model as discussed above) to data of patient 102. Data of patient 102 may include HRV features (e.g., as determined based on heart rate data 110) and/or data corresponding to clinical features of patient 102. External device 108 may receive the data of patient 102 from IMD 106, one or more EHR system 114, HMS 116, and/or patient 102, e.g., via UI 308.

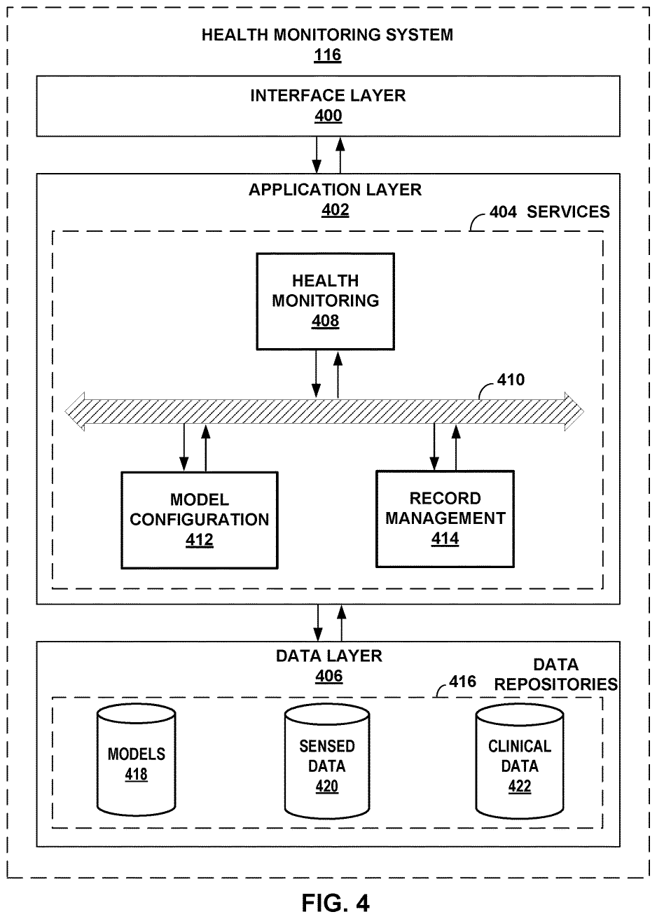

FIG. 4 is a block diagram illustrating an example health monitoring system (HMS) 116 of the medical device system 100 of FIG. 1. HMS 116 may be implemented in one or more computing devices (e.g., external device 108), one or more computing systems, and/or a cloud computing environment, and may include hardware components such as those of external device 108, e.g., processing circuitry, memory, and communication circuitry, embodied in one or more physical devise. FIG. 4 provides an operating perspective of HMS 116 when hosted as a cloud-based platform. In the example of FIG. 4, components of HMS 116 are arranged according to multiple logical layers that implement the techniques of this disclosure. Each layer may be implemented by one or more modules comprised of hardware, software, or a combination of hardware and software.

Computing devices and/or systems, such as external device 108 and network 112, operate as clients that communicate with HMS 116 via interface layer 400. The computing devices and/or systems typically execute client software applications, such as desktop application, mobile application, and web applications. Interface layer 400 represents a set of application programming interfaces (API), or protocol interfaces presented and supported by HMS 116 for the client software applications. Interface layer 400 may be implemented with one or more web servers.

As shown in FIG. 4, HMS 116 also includes an application layer 402 that represents a collection of services 404 for implementing the functionality ascribed to HMS 116 herein. Application layer 402 receives information from client applications e.g., heart rate data 110, and/or data on HRV features and/or clinical features, from external device 108 and/or network 112, and further processes the information according to one or more of the services 404 to respond to the information. Application layer 402 may be implemented as one or more discrete software services 402 executing on one or more application servers, e.g., physical or virtual machines. That is, the application servers provide runtime environments for execution of services 402. In some examples, the functionality interface layer 400 as described above and the functionality of application layer 402 may be implemented at the same server. Services 404 may communicate via a logical service bus 410. Service bus 410 generally represents a logical interconnection or set of interfaces that allows different services 404 to send messages to other services, such as by a publish/subscription communication model.

Data layer 406 of HMS 116 provides persistence for information in medical device system 100 using one or more data repositories 416. A data repository 416, generally, may be any data structure or software that stores and/or manages data. Examples of data repositories 416 include but are not limited to relational databases, multi-dimensional databases, maps, and hash tables, to name only a few examples. Data repository 416 may include, but are not limited to, models 418, sensed data 420, and clinical data 422. Sensed data 420 may include data of patient 102 and/or other patients corresponding to HRV features. Clinical data 422 may include data of patient 102 and/or other patients corresponding to clinical features.

As shown in FIG. 4, each of services 408, 412, and 414 is implemented in a modular form within HMS 116. Although shown as separate modules for each service, in some examples the functionality of two or more services may be combined into a single module or component. Each of services 408, 412, and 414 may be implemented in software, hardware, or a combination of hardware and software. Moreover, services 408, 412, and 414 may be implemented as standalone devices, separate virtual machines or containers, processes, threads, or software instructions generally for execution on one or more physical processors.

Health monitoring service 408 may monitor and record data on patient 102. The data on patient 102 may include, but are not limited to, heart rate data 110, data on HRV features of patient 102, or data on clinical features of patient 102. Health monitoring service 408 may monitor and record the data automatically or based on user inputs, e.g., through external device 108 and network 112. Health monitoring service 408 may receive the data (e.g., via network 112) from one or more of external device 108, EHS system 114, or one or more other computing devices, computing systems, or cloud computing environments connected to network 112.

Record management service 414 may store the data recorded by health monitoring service 408 within data repositories 416 (e.g., in sensed data repository 420, in clinical data repository 422, or the like). Rules configuration service 412 may determine the one or more models based on data stored in data repositories 416. Rules configuration service 412 may train the ML models using information retrieved from data repositories 416. data repositories 416 may contain data on the HRV features and the clinical features of patient 102 and/or other patients.

Each ML model may include one or more classification algorithms (also referred to as “classifiers”). Each of the one or more classifiers may be configured to generate a predicted effect of the medical procedure based on the inputs (e.g., data on patient 102). The classifiers may include single classifiers, which uses a single model to generate a prediction, and/or ensemble classifiers, which may combine multiple models to generate a prediction. The single classifiers may include, but are not limited to, support vector machines with linear kernels (SVM), polynomial kernels (SVMp), or gaussian kernels (SVMg). The ensemble classifiers may include, but are not limited to, classification and regression trees (CART) or K-nearest neighbor analysis (KNN).

Each of the ML models may include weight values corresponding to each of the classifiers included in the ML model. During application, the ML model may determine a predicted effect of a medical procedure based on the predicted effect of each of the classifiers and the weight values assigned to each of the classifiers of the ML model. For example, when applying a ML model, processing circuitry 302 may give greater weight to a particular classifier based on the weight value assigned to the classifier. During training of the ML model, rules configuration service 412 may assign weight values to each of the classifiers. In some examples where the predicted effects of a medical procedure are binary (e.g., recurrence of a medical condition or non-recurrence of the medical condition), the predictions of each of the classifiers is represented by a corresponding voting vector (e.g., a voting vector of 1 for a prediction of recurrence or a voting vector of 0 for a prediction of non-recurrence).

Rules configuration service 412 may, as a part of training the ML model, select one or more features from the HRV features and the clinical features (collectively referred to as “available features”). The ML model may include a plurality of slots, each of the plurality of slots configured to accept a feature from the available features. ML model may be configured to, for each of the classifiers in the ML model, input data corresponding to each of the features contained in the plurality of slots into the classifier to generate a respective predicted effect of the medical procedure.

Rules configuration service 412 may, as a part of training the ML model, in separate stages, including one or more forward selection stages, one or more classification stages, and one or more backward selection stages. The forward selection stage may be configured to select features from the available features and determine an optimal placement of the selected feature within the plurality of slots within the ML model. In some examples, the one or more forward selection stages may include application of a Sequential Forward Floating Search (SFFS) algorithm.

As a part of the forward selection stage, model configuration module 412 may select a first feature from the available features and place the first feature into a first slot of the plurality of slots. As a part of the classification stage, model configuration module 412 may determine a range of possible weight values for each of the classifiers in ML model. In some examples, processing circuitry 302 assigns equal weight to all of the classifiers in the ML model (also referred to as “mean voting method”). In some examples, model configuration module 412 assigns weight to each of the classifiers based on the accuracy of the classifier (also referred to as “accuracy weighted voting method”). In some examples, model configuration module 412 assigns weight to each of the classifiers based on a plurality of weighting configurations with different weight steps (e.g., in weight steps of 0.1) (also referred to as “optimum weighted voting method”). Model configuration module 412 may determine the range of possible weight values based on one or more of the mean voting method, the accuracy weighted voting method, or the optimum weighted voting method.

Model configuration module 412 may apply input data from prior patients on the selected feature into every combination of possible weight values from the range of possible weight values and position of the selected feature in each of the plurality of slots to determine an optimal combination of weight values and placement of the selected feature within the ML model. In some examples, the optimal combination for the selected feature is a combination of the weight values and placement of the selected feature that maximizes the accuracy of the ML model with regard to the selected feature. Model configuration module 412 may iteratively perform the forward selection stage and classification stage to select features and place features into available slots in the plurality of slots of the ML model until a threshold number of features are selected. The threshold number of selected features may be four or more features.

During the backward selection stage, model configuration module 412 may remove selected features that reduce the accuracy of the ML model from the plurality of slots. In some examples, during the backward selection stage, model configuration module 412 iteratively removes each of the selected features in the ML model and determines the accuracy of the ML model without the removed feature. If model configuration module 412 determines that the removal of the feature increases the accuracy of the ML model, model configuration module 412 may permanently remove the feature from the ML model and return to the backward selection stage and/or the forward selection stage. If model configuration module 412 determines that the removal of the feature doesn't increase the accuracy of the ML mode, model configuration module 412 may leave the feature in the ML model and/or may designate the feature as a validated feature. In some examples, model configuration module 412 performs the backward selection stage on the ML model based on a determination that ML model contains the threshold number of selected features. Model configuration module 412 may train the ML model by iteratively applying the forward selection stage, classification stage, and backward selection stage, e.g., until the ML model satisfies a threshold average accuracy for the predicted effects. Once model configuration module 412 determines that the ML model is trained, HMS 116 may store the ML model in data repositories 416, e.g., in models 418 of data repositories 416. In some examples HMS 116 may transmit the trained ML model to external device 108 for storage in memory 304 of external device 108, e.g., in model engine module 314. Processing circuitry 302 may then execute computer-readable instructions stored in model engine module 314 corresponding to the trained ML model to apply the model to the data on patient 102 (e.g., data on HRV features and/or clinical features) to predict an effect of a particular medical procedure on patient 102.

FIG. 5 is a conceptual diagram illustrating an example set 500 of heart rate data 110 recorded by an IMD 106 of the medical device system 100 of FIG. 1. While FIG. 5 is described with heart rate data 110 represented as R-R intervals over time and with AF as the medical condition. In other examples, other representations of heart rate data 110 may be used for other types of medical conditions.

FIG. 5 illustrates heart rate data 110 corresponding to onset of a medical condition (an AF episode 508, as illustrated in FIG. 5) and a flashback period 502 immediately preceding the onset. Flashback period 502 may be separated into a first flashback period 504 and a second flashback period 506. First flashback period 504 may represent a first set number (e.g., 100, 200, 300, or the like) of R-R intervals in flashback period 502. Second flashback period 506 may represent a second set number (e.g., 100, 200, or the like) of R-R intervals immediately preceding AF episode 508. As FIG. 5 illustrates, there may be short-term changes in the duration of R-R intervals in flashback period 502 preceding AF episode 508. For example, R-R intervals in first flashback period 504 are relatively longer than R-R intervals in second flashback period 506.

In some examples, medical device system 100 determines HRV features from heart rate data 110 by determining HRV features for one or more of flashback period 502, first flashback period 504, second flashback period 506, and AF episode 508. In some examples, medical device system 100 determines Mean value, pNNX, RMSSD, SDNN, TINN, TRI, ApEn, SampEn, SD1, SD2, SD1:SD2 ratio, DFA α1, and/or DFA α2 for one or more of flashback period 502, first flashback period 504, second flashback period 506, or AF episode 508. During training of the ML models, medical device system 100 may select HRV features corresponding to any or all of flashback period 502, first flashback period 504, second flashback period 506, or AF episode 508.

FIG. 6 is a conceptual diagram illustrating an example neural network 600 configured to predict the effects of the medical procedure. While FIG. 6 describes the model including neural network 600, other example models described herein may include other ML and/or non-ML techniques and models. Neural network 600 may include an input layer 602, hidden layer 604, and an output layer 606. Neural network 600 may be stored in memory of one or more computing devices, computing systems, and/or cloud computing environments (e.g., in models 322 and/or models 418).

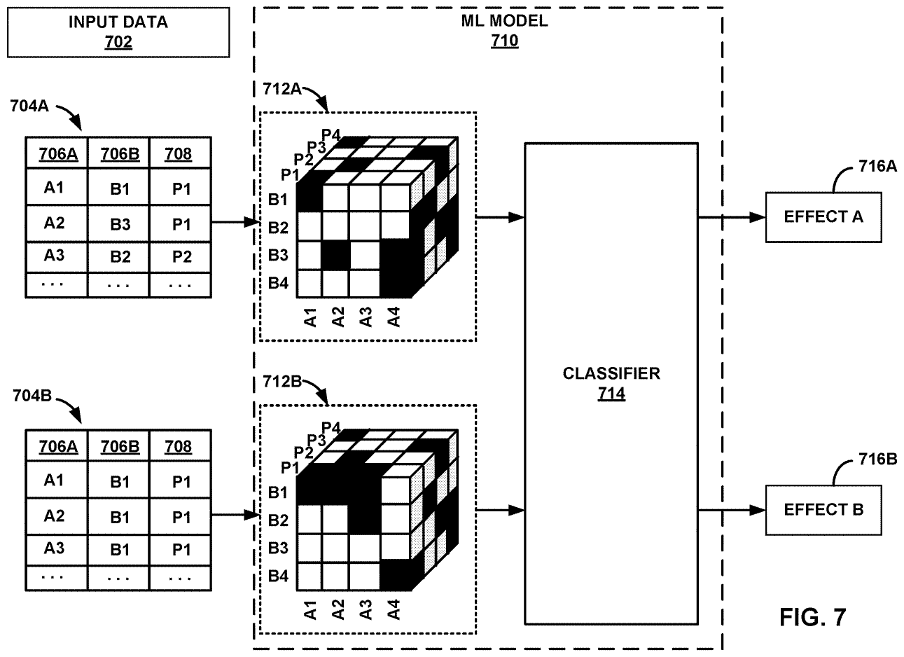

Input layer 602 includes inputs 608A-D (collectively referred to as “inputs 608”). Each of inputs 608 may represent a source of data input into neural network 600. In some examples, each of inputs 608 may represent a distinct HRV feature or clinical feature. In some examples, each of inputs 608 may represent a combination of HRV features and/or clinical features, e.g., as described in greater detail in FIG. 7.

Input layer 602 may be connected to hidden layer 604 and inputs 608 may be transmitted to hidden layer 604. Hidden layer 604 may include layers 610A-N (collectively referred to as “layers 610”), each of layers 610 including one or more nodes 612. Hidden layer 604 may weigh and/or aggregate inputs 608 to produce an output (e.g., a predicted effect of a medical procedure) based on the input data. The structure of hidden layer 604 (e.g., number of layers 610, number of nodes 612, disposition of pathways between nodes 612) and/or the functions of hidden layer 604 (e.g., the manner of aggregation of inputs 608, the manner of weighing of inputs 608) may vary based on the desired output. In some examples, one or more computing devices, computing systems, and/or cloud computing environments (e.g., external device 108, HMS 116, or the like) may determine and/or adjust the number of layers 610, the structure of each layer 610, the weighing of each of inputs 608, and/or the manner of aggregation of inputs 608 via one or more ML training techniques, e.g., as previously discussed herein.

Hidden layer 604 may determine, based on inputs 608 and functions performed by hidden layer 604, outputs 614A-B (collectively referred to as “outputs 614”) of output layer 606. Outputs 614 may include, but are not limited to, the probability of occurrence one or more medical conditions within a certain period. For example, output 614A may correspond to the probability of occurrence of an AF episode within a certain period after the medical procedure and output 614B may correspond to the probability of occurrence of another illness within a certain period after the medical procedure.

FIG. 7 is a conceptual diagram illustrating an example process of inputting data into an example model for the prediction of the effects of the medical procedure. While FIG. 7 illustrated the example model as a ML model 710, other examples may include rule-based model (e.g., rule-based expert systems).

Input data 702 for ML model 710 may be represented as multiway arrays 704A-B (collectively referred to as “multiway arrays 704”). Each of multiway arrays 704 may store a plurality of inputs 706A-B (collectively referred to as “inputs 706”). Each of inputs 706 may represent a HRV feature or a clinical feature. Each of multiway arrays 704 may store the values of inputs 706 and the position of the values of input 706 within a corresponding tensor representation of tensor representations 712A-B (collectively referred to as “tensor representations 712”).

The position of particular values within the corresponding tensor representation 712 may be represented by the markers (e.g., markers A1-A4, B1-B4) and ports 708 stored in multiway arrays 704. Each of the plurality of markers may have a respective value of the feature represented by the corresponding input of inputs 706. Each of ports 708 may represent a point of data input (e.g., inputs 608) into classifier 714. For each combination of inputs 706A and 706B stored in multiway arrays 704, the corresponding tensor representation 712 may represent the combination of the values of the inputs 706 via a vector. As illustrated in FIG. 7, depending on the desired output, the combinations of inputs 706 and placements of the combinations of inputs 706 within tensor representation 712B may be different.

Multiway arrays 704 may be represented as tensor representations 712 which may be inputted into classifier 714 of ML model 710 to generate corresponding outputs 716A-B (collectively referred to as “outputs 716”). Each of outputs 716 may each represent a likelihood of occurrence of a particular predicted effect of a medical procedure. While the tensor representations 712 illustrated in FIG. 7 are third order tensors, other examples may include first order, second order, or fourth order or higher tensors as inputs to an example model.

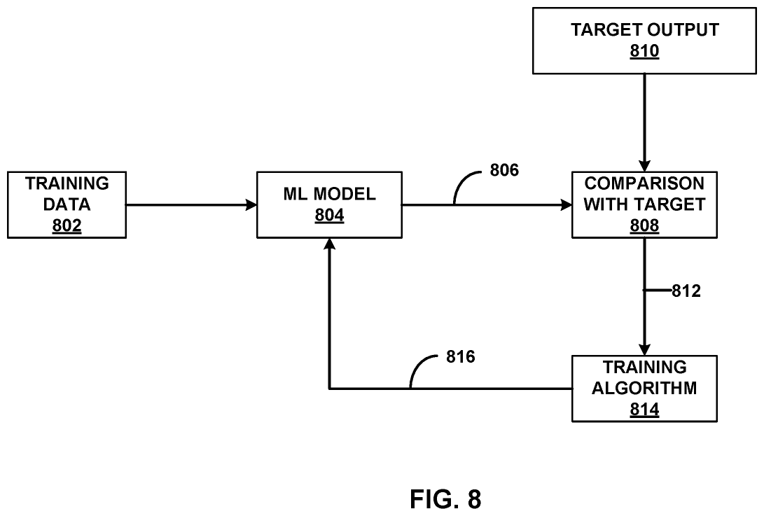

FIG. 8 is a block diagram illustrating an example process of training an example model for the prediction of the effects of the medical procedure. While FIG. 8 is described primarily with reference to a computing system, the example process described may be performed using one or more computing devices, computing systems, and/or cloud computing environments (e.g., external device 108, HMS 116, or the like). A computing system may generate ML model 804 (e.g., with randomly assigned weights and/or structure). A computing system may input training data 802 into ML model to generate prediction 806. Training data 802 may include respective values for one or more HRV features and/or clinical features. In some examples, the respective values for the one or more features may include sensed data for the one or more features, e.g., from patient 102 and/or one or more other persons. The respective values may be organized into training instances of a training set of training data 802. In some examples, each training instance may correspond to the respective values for the HRV features and the clinical features of a single person after the medical procedure. In some examples, each training instance may correspond to aggregated values for HRV features and clinical features of a plurality of similar individuals who had undergone the medical procedure. In some examples, multiple training instances may correspond to a single individual, each of the multiple training instance corresponding to the respective values for the HRV features and the clinical features of the individual after a respective medical procedure of a plurality of medical procedures undergone by the individual. In some examples, the computing system generates one or more training instances of the set of training instances from the one or more heart rate variability features and the one or more clinical features after the computing system receives the corresponding determined effect of the medical procedure (e.g., via IMD 106, UI 308 of external device 108, or the like).

The computing system may perform comparison 808 between prediction 806 and target output 810. Target output 810 may include determined effects of a medical procedure corresponding to the training instances of training data 802. The computing system may determine, based on comparison 808, error data 812 between prediction 806 and target output 810. In some examples, the computing system may for each training instance in the training set, modify, based on particular values and a particular determined effect of the medical procedure, the model to change a likelihood predicted by the model for the particular predicted effect associated with the particular values in response to subsequent values for the one or more of the one or more heart rate variability features or the one or more clinical features applied to the model.

Based on the error data 812, the computing system may apply a training algorithm 814 (also referred to as “learning algorithm 814”) to adjust weights and/or structure of ML model 804. The computing system may then transmit adjustments 816 to ML model 804 and adjust ML model 804 based on adjustments 816. The computing system may then input subsequent data from training data 802 and perform the process until predictions 806 differ from target output 810 by less than a predetermined amount (e.g., by less than a predetermined percentage).

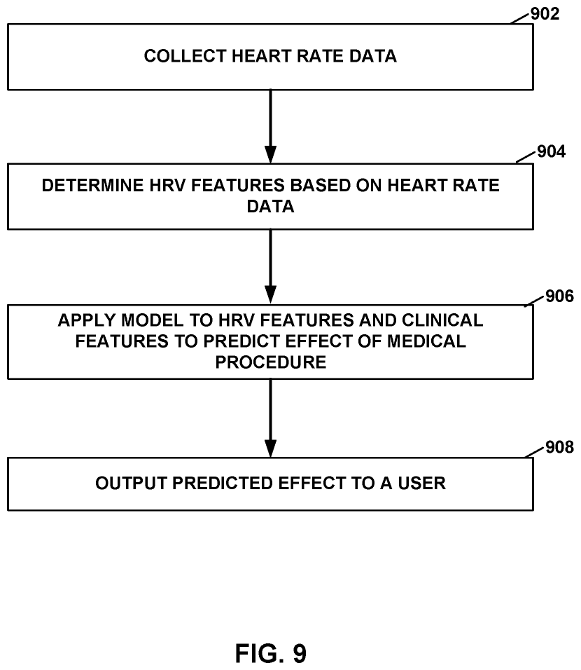

FIG. 9 is a flowchart illustrating an example process of predicting effects of a medical procedure. A medical device system (e.g., medical device system 100 of FIG. 1) may collect heart rate data 110 from heart 104 of patient 102 (902). In some examples, an ID (e.g., IMD 106) of medical device system 100 collects heart rate data 110 via one or more electrodes (e.g., electrodes 202) electrically connected to heart 104 of patient. IMD 106 may collect heart rate data 110 by monitoring and recording heart rate data 110, e.g., in accordance with the example process discussed with respect to IMD 106 in FIG. 2. In some examples, heart rate data 110 is collected as QRS complexes corresponding to ventricular depolarizations of heart 104. In some examples IMD 106 identifies R-waves within the collected QRS complexes.

Medical device system 100 may determine HRV (HRV) features based on heart rate data 110 (904). External device 108, HMS 116, and/or one or more computing devices, computing systems, and/or cloud computing environments connected to network 112 of medical device system 110 may be configured to determine the HRV features. HRV features may include, but are not limited to: Mean value, pNNX, RMSSD, SDNN, TINN, TRI, ApEn, SampEn, SD1, SD2, SD1:SD2 ratio, DFA α1, and/or DFA α2. The HRV features may be determine for one or more of flashback period 502, first flashback period 504, second flashback period 506, or AF episode 508, e.g., as illustrated in FIG. 5. In some examples, the HRV features are further stored in external device 108, HMS 116, EHS system 114, and/or one or more computing devices, computing systems, and/or cloud computing environments connected to network 112.

Medical device system 100 may apply a model to HRV and clinical features predict the effect of a medical procedure (906). The effects of a medical procedure may include efficacy of the procedure and likelihood of recurrence of the target medical condition. In some examples the predicted effects of the medical procedure include predicted recurrence and/or non-recurrence of the target medical condition (e.g., AF) within a set period (e.g., within 12 months after the performance of the medical procedure. Medical device system 100 may apply the model by inputting data of patient 102 corresponding to features from HRV features and/or clinical features that are disposed within the model and outputting a predicted result based on the application of the model. In some examples, medical device system 100 may input determined values for the one or more HRV features and the one or more clinical features into the model and generate, using the model and based on the inputted determined values, a plurality of possible effects of the medical procedure. Medical device system 100 may determine, using the model a respective likelihood of occurrence for each possible effect of the plurality of possible effects and select a predicted effect from the plurality of possible effects. Medical device system 100 may select the predicted effect from the plurality of possible effects based on the corresponding likelihoods of occurrence. In some examples, medical device system 100 may select the possible effect with the highest likelihood of occurrence as the predicted effect.

Medical device system 100 may output the predicted effect to a user (908). In some examples, the user includes a medical professional and/or patient 102. Medical device system 100 may output the predicted effect to a user interface (e.g., UI 308) of external device 108 or to one or more separate display devices connected to network 112. The outputted information to the user may include but is not limited to, the predicted effect (e.g., of a recurrence/non-recurrence of the medical condition), the features medical device system 100 used to determine the predicted effect, the likelihood (e.g., in percentages) of the predicted effect, the prediction of each of one or more classifiers used by medical device system 100 to determine the predicted effect, or the like.

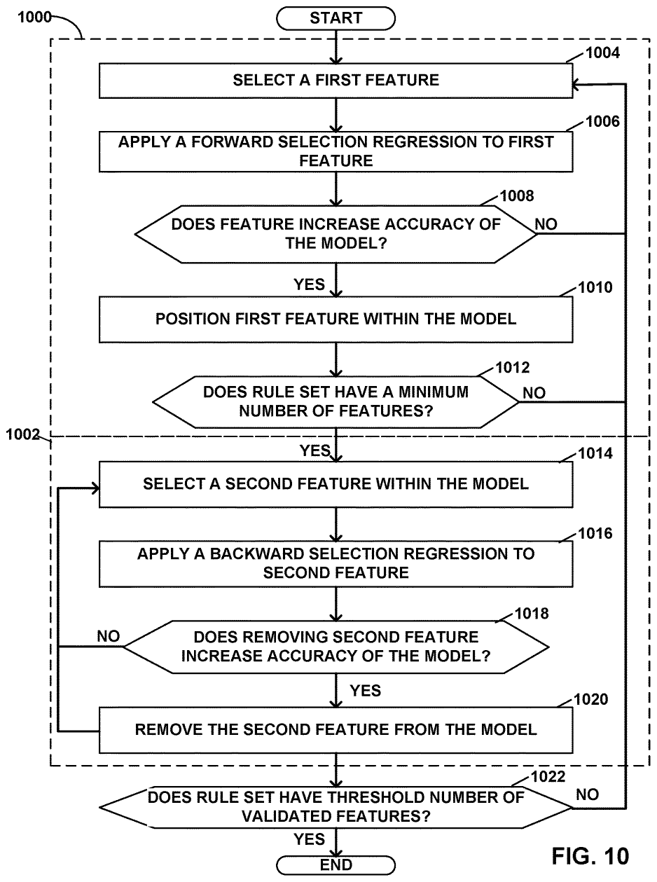

FIG. 10 is a flowchart illustrating an example process of generating a model for the prediction of the effects of the medical procedure. Specifically, FIG. 10 describes an example process for generating a model by training a ML model. Medical device system 100 may generate the model by applying a forward selection stage 1000 and a backward selection stage 1002 to select features for the model. Forward selection stage 1000 may include a classification stage.

In forward selection stage 1000, medical device system 100 may select a first feature from a plurality of features (1004). The plurality of features may include HRV features and/or clinical features. HRV features may include HRV features for flashback period 502, first flashback period 504, second flashback period 506, or AF episode 508, e.g., as described in FIG. 5. Medical device system 100 may apply a forward selection regression to the first feature (1006). The forward selection regression may include a SFFS algorithm. As a part of the forward selection regression, medical device system 100 may apply a classification stage to select weight values from a range of weight values for each of one or more classifiers in the model. The weight values may represent how much weight medical device system 100 should give to each of predicted effects of the classifiers in determining an overall predicted effect. Medical device system 100 may generate the range of weight values using one or more methods including, but are not limited to, mean voting method, accuracy weighted voting method, or optimum weighted voting method. Medical device system 100 may, for each of the classifiers, select a weight value that maximizes the accuracy of the predictions of the classifier. Medical device system 100 may, as a part of applying the forward selection regression to the first feature, determine an optimal placement of the first feature within a plurality of feature slots of the model, wherein the optimal placement of the first feature may be a slot of the plurality of slots that maximizes the accuracy of the model. Medical device system 100 may determine weight values of the classifiers and the optimal placement of the first feature by using data of prior patients as inputs to the model and comparing the predicted effects of the model to the actual effects of the medical procedure on the prior patients.

Medical device system 100 may determine, (e.g., based on the results of the forward selection regression) if the first feature increases the accuracy of the model (1008). If the first feature does not increase the accuracy (“NO” branch of 1008), medical device system 100 may reject the first feature and select a new feature from the plurality of features (1004). If the first feature increases the accuracy (“YES” branch of 1008), medical device system 100 may position the first feature within the model, e.g., at the optimal placement within the model (1010).

Medical device system 100 may determine if the model includes a minimum number of features (1012). If the model does not include the minimum number of features (“NO” branch of 1012), medical device system 100 may iteratively perform forward selection stage 1000 (e.g., steps 1004-1010) until the model includes the minimum number of features. If the model includes the minimum number of features (“YES” branch of 1012), medical device system 100 may proceed to backward selection stage 1002 (e.g., step 1014).

During the backward selection stage 1002, medical device system 100 may select a second feature within the model (1014). Medical device system 100 may apply a backward selection regression to the second feature (1016). Medical device system 100 may, as a part of applying the backward selection regression, remove the second feature from the model and determine the accuracy of the model without the second feature. Based on a determination that removing the second feature increases the accuracy of the model (“YES” branch of 1018), medical device system 100 removes the second feature from the model (1020) and selects a new feature within the model (1014) to apply backward selection stage 1002. Based on a determination that removing the second feature does not increase the accuracy of the model (“NO” branch of 1018), medical device system 100 re-places the second feature within the model and selects a new, different feature within the model (1014) to apply backward selection stage 1002. Medical device system 100 may also designate the second feature as a validated feature.

Medical device system 100 may determine if the model satisfies a threshold accuracy condition (1022). In some examples, the threshold accuracy condition may be predetermined. In some examples, the threshold accuracy condition is a maximum achievable accuracy for the model. If medical device system 100 determines that the model satisfies the threshold accuracy condition (“YES” branch of 1022), medical device system 100 may complete the model generation process. If medical device system 100 determines that the model does not satisfy the threshold accuracy condition (“NO” branch of 1022), medical device system 100 may select new features from the plurality of features (1004) and repeat forward selections stage 1000 and backward selection stage 1002. Medical device system 100 may repeat the example process described above until the model satisfies the threshold accuracy condition.

The devices, systems, and techniques of this disclosure provides improvements over other prediction systems. The incorporation of HRV features and clinical features from a medical device enables improved prediction of recurrences of medical conditions in the heart of the patient prior to performance of the medical procedure. In some examples, this disclosure further describes generation of the models used in the prediction the effects of the medical procedures. The models may incorporate portions of HRV features and clinical features to increase the accuracy of the predictions on the effects of the medical procedures.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various modules and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. For example, any module described herein may include electrical circuitry configured to perform the features attributed to that particular module, such as fixed function processing circuitry, programmable processing circuitry, or combinations thereof.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

It should be noted that medical device system 100, and the techniques described herein, may not be limited to use in a human patient. In alternative examples, medical device system 100 may be implemented in non-human patients, e.g., primates, canines, equines, pigs, and felines. These other animals may undergo clinical or research therapies that my benefit from the subject matter of this disclosure. Various examples are described herein, such as the following examples.

Example 1: a computing system comprising: memory configured to store heart rate data; a display device; and processing circuitry configured to: collect heart rate data of the patient from a medical device of the patient; determine one or more heart rate variability features based on the heart rate data; apply a model to the one or more heart rate variability features and one or more clinical features of the patient; predict an effect of a medical procedure on the patient based on the application of the model to the one or more heart rate variability features and the one or more clinical features; and output the predicted effect of the medical procedure to the display device.

Example 2: the computing system of example 1, wherein the heart rate data comprises a plurality of time intervals corresponding to time periods between electrical signals recorded by the medical device, the electrical signals corresponding to depolarizations of a first chamber of a heart of the patient.

Example 3: the computing system of example 2, wherein the electrical signals comprise QRS complexes detected by the medical device, and wherein the time periods comprise times between R-waves of adjacent QRS complexes.

Example 4: the computing system of any of examples 1-3, wherein the one or more heart rate variability features comprises: an average value of the plurality of time intervals; a mean square difference of adjacent time intervals of the plurality of time intervals; a standard deviation of the plurality of time intervals; or a percentage of the plurality of time intervals that satisfy a threshold time condition.

Example 5: the computing system of any of examples 1-4, wherein the one or more clinical features of the patient comprises one or more of: an age of the patient; a presence of an illness in the patient; or a length of a monitoring period of the patient prior to a past medical procedure.

Example 6: the computing system of examples 5, wherein the illness comprises one or more of: paroxysmal atrial fibrillation; hypertension; diabetes; coronary artery disease; lesions; or stroke.

Example 7: the computing system of any of examples 5 and 6, wherein the past medical procedure comprises a cardiac ablation procedure.

Example 8: the computing system of any of examples 1-7, wherein the medical device comprises an implantable cardiac monitor (ICM).

Example 9: the computing system of any of examples 1-8, wherein the effect of a medical procedure on the patient comprises a recurrence of an atrial fibrillation (AF) episode experienced by the patient after performance of the medical procedure on the patient.

Example 10: the computing system of example 9, wherein the predicted effect of the medical procedure comprises a probability of the recurrence of the AF episode within a set period after the performance of the medical procedure on the patient.

Example 11: the computing system of example 10, wherein the set period comprises 12 months after the performance of the medical procedure.

Example 12: the computing system of any of examples 1-11, wherein the medical procedure comprises a cardiac ablation procedure.

Example 13: the computing system of any of examples 1-12, wherein to apply the model, the processing circuitry is further configured to determine the model, and wherein to determine the model, the professing circuitry is further configured to: select a first feature of the one or more heart rate variability features and the one or more clinical features for a prediction module; determine a weight value for the first feature based on a plurality of classifier modules; and generate a second prediction module comprising the first feature and the weight value.

Example 14: the computing system of example 13, wherein to select the first feature, the processing circuitry is configured to apply a sequential forward floating search (SFFS) to the one or more heart rate variability features and the one or more clinical features, and wherein to apply the SFFS, the processing circuitry is configured to: select a first feature from the one or more heart rate variability features and the one or more clinical features; apply a forward selection regression to the prediction module and the first feature; and position the first feature within a position in the prediction module that maximizes the accuracy of the prediction module.

Example 15: the computing system of any of examples 13 and 14, wherein to generate the second prediction module, the processing circuitry is further configured to: select a second feature from a plurality of existing features in the prediction module; apply a backward selection regression to the prediction module and the second feature; and remove, based on a determination that removing the second feature increases the accuracy of the prediction module, the second feature from the prediction module.

Example 16: the computing system of any of examples 13-15, wherein to determine the weight value, the processing circuitry is configured to: apply a weighted voting system a to a plurality of weight vectors and a plurality of corresponding voting vectors, wherein each weight vector and the corresponding voting vector corresponds to one of the plurality of classifier modules; and determine an accuracy of the prediction module associated with each of the plurality of weight vectors and the plurality of corresponding voting vector.

Example 17: the computing system of example 16, wherein at least one of the plurality of classifier modules comprises a machine learning model.

Example 18: the computing system of any of examples 13-17, wherein the plurality of classifier modules comprises at least five classifier modules.

Example 19: the computing system of any of examples 1-18, wherein the processing circuitry is configured to generate the model, and wherein to generate the model, the processing circuitry is configured to: select a training set comprising a set of training instances, each training instance comprising an association between respective values for one or more of the one or more heart rate variability features or the one or more clinical features and a determined effect of the medical procedure; and for each training instance in the training set, modify, based on particular values and a particular determined effect of the medical procedure, the model to change a likelihood predicted by the model for the particular predicted effect associated with the particular values in response to subsequent values for the one or more of the one or more heart rate variability features or the one or more clinical features applied to the model.

Example 20: the computing system of example 19, wherein the respective values comprises sensed values for the one or more of the one or more heart rate variability features or the one or more clinical features from one or more other persons.

Example 21: the computing system of any of examples 19 and 20, wherein the processing circuitry is configured to generate one or more training instances of the set of training instances from the one or more heart rate variability features and the one or more clinical features after the computing system receives the corresponding determined effect of the medical procedure.

Example 22: the computing system of any of examples 1-21, wherein to predict the effect of the medical procedure on the patient based on the application of the model to the one or more heart rate variability features and the one or more clinical features, the processing circuitry is configured to: input determined values for the one or more heart rate variability features and the one or more clinical features into the model; generate, using the model and based on the inputted determined values, a plurality of possible effects of the medical procedure; determine, using the model, a respective likelihood of occurrence for each respective possible effect of the plurality of possible effects; and select, based on the respective likelihoods of occurrence, a predicted effect of the medical procedure from the plurality of possible effects.

Example 23: a method comprising: collecting, by a computing system, heart rate data of a patient from a medical device of the patient; determining, by the computing system, one or more heart rate variability features based on the heart rate data; applying, by the computing system, a model to the heart rate variability features and one or more clinical features of the patient; predicting, by the computing system, an effect of a medical procedure on the patient based on the application of the model to the heart rate variability features and the one or more clinical features; and outputting, by the computing system, the predicted effect of the medical procedure to a display device.

Example 24: the method of example 23, wherein the heart rate data comprises a plurality of time intervals, the plurality of time intervals corresponding to electrical signals recorded by the medical device, the electrical signals corresponding to depolarization of a first chamber of a heart of the patient.

Example 25: the method of example 24, wherein the electrical signals comprise QRS complexes detected by the medical device, and wherein the time periods comprise times between R-waves of adjacent QRS complexes.

Example 26: the method of any of examples 24 and 25, wherein the one or more heart rate variability features comprises one or more of: an average value of the plurality of time intervals; a mean square difference of adjacent time intervals of the plurality of time intervals; or a standard deviation of the plurality of time intervals.

Example 27: the method of any of examples 24-26, wherein the one or more heart rate variability features comprises a percentage of the plurality of time intervals that satisfy a threshold time condition.

Example 28: the method of example 27, wherein the threshold time condition is between 10 milliseconds (ms) and 70 ms.

Example 29: the method of any of examples 23-28, wherein the one or more clinical features of the patient comprises one or more of: an age of the patient; a presence of an illness in the patient; or a length of a monitoring period of the patient prior to a past medical procedure.

Example 30: the method of example 29, wherein the illness comprises one or more of: paroxysmal atrial fibrillation; hypertension; diabetes; coronary artery disease; lesions; or stroke.

Example 31: the method of any of examples 29 and 30, wherein the past medical procedure comprises a cardiac ablation procedure.

Example 32: the method of any of examples 23-31, wherein the medical device comprises an implantable cardiac monitor (ICM).

Example 33: the method of any of examples 23-32, wherein the effect of a medical procedure on the patient comprises a recurrence of an atrial fibrillation (AF) episode experienced by the patient after performance of the medical procedure on the patient.

Example 34: the method of example 33, wherein the predicted effect of the medical procedure comprises a probability of the recurrence of the AF episode within a set period after the performance of the medical procedure on the patient.

Example 35: the method of example 34, wherein the set period comprises 12 months after the performance of the medical procedure.

Example 36: the method of any of examples 23-35, wherein the medical procedure comprises a cardiac ablation procedure.

Example 37: the method of any of examples 23-36, further comprising determining the model by at least: selecting, by the computing system, a first feature of the one or more heart rate variability features and the one or more clinical features for a prediction module; determining, by the computing system, a weight value for the first feature based on a plurality of classifier modules; and generating, by the computing system, a second prediction module comprising the first feature and the weight value.

Example 38: the method of example 37, wherein selecting the first feature comprises applying, by the computing system, sequential forward floating search (SFFS) to the one or more heart rate variability features and the one or more clinical features, wherein applying the SFFS comprises: selecting, by the computing system, the first feature from the one or more heart variability features and the one or more clinical features; applying, by the computing system, a forward selection regression to the prediction module and the first feature; and positioning, by the computing system, the first feature within a position in the prediction module that maximizes the accuracy of the prediction module.

Example 39: the method of any of examples 37 and 38, wherein generating the second prediction module further comprises: selecting, by the computing system, a second feature from a plurality of existing features in the prediction module; applying, by the computing system, a backward selection regression to the prediction module and the second feature; and based on a determination that removing the second feature increases the accuracy of the prediction module, removing, by the computing system, the second feature from the prediction module.

Example 40: the method of any of examples 37-39, wherein determining the weight value comprises: applying, by the computing system, a weighted voting system to a plurality of weight vectors and a plurality of corresponding voting vectors, wherein each weight vector and the corresponding voting vector corresponds to one of the plurality of classifier modules; and determining an accuracy of the prediction module associated with each of the plurality of weight vectors and the plurality of corresponding voting vector.

Example 41: the method of any of examples 37-39, wherein determining the weight value comprises: applying one or more of the plurality of classifier modules to the one or more heart rate variability features and the one or more clinical features; and determining an accuracy of the prediction module associated with each of the plurality of weight vectors and the plurality of corresponding voting vector.

Example 42: the method of claim 41, wherein at least one of the plurality of classifier modules comprises a machine learning model.

Example 43: the method of any of examples 37-42, wherein the plurality of classifier modules comprises at least five classifier modules.

Example 44: the method of any of examples 23-43, wherein further comprising generating, by the computing system, the model, and wherein generating the model comprises: selecting, by the computing system, a training set comprising a set of training instances, each training instance comprising an association between respective values for one or more of the one or more heart rate variability features or the one or more clinical features and a determined effect of the medical procedure; and for each training instance in the training set, modifying, by the computing system and based on particular values and a particular determined effect of the medical procedure, the model to change a likelihood predicted by the model for the particular predicted effect associated with the particular values in response to subsequent values for the one or more of the one or more heart rate variability features or the one or more clinical features applied to the model.

Example 45: the method of example 44, wherein the respective values comprise sensed values for the one or more of the one or more heart rate variability features or the one or more clinical features from one or more other persons.

Example 46: the method of any of examples 44 and 45, further comprising generating, by the computing system, one or more training instances of the set of training instances from the one or more heart rate variability features and the one or more clinical features after the computing system receives the corresponding determined effect of the medical procedure.

Example 47: the method of any of examples 23-46, wherein predicting, the effect of the medical procedure on the patient based on the application of the model to the heart rate variability features and the one or more clinical features comprises: inputting, by the computing system, determined values for the one or more heart rate variability features and the one or more clinical features into the model; generating, by the computing system and using the model and based on the inputted determined values, a plurality of possible effects of the medical procedure; determining, by the computing system and using the model, a respective likelihood of occurrence for each respective possible effect of the plurality of possible effects; and selecting, by the computing system and based on the respective likelihoods of occurrence, a predicted effect of the medical procedure from the plurality of possible effects.

Example 48: a computer readable storage medium comprising instructions that, when executed, cause processing circuitry within a device to perform the method of any of examples 23-47.

Various examples have been described herein. Any combination of the described operations or functions is contemplated. These and other examples are within the scope of the following claims.

1. A computing system comprising:
memory configured to store heart rate data;
a display device; and
processing circuitry configured to:
collect heart rate data of the patient from a medical device of the patient;
determine one or more heart rate variability features based on the heart rate data;
apply a model to the one or more heart rate variability features and one or more clinical features of the patient;
predict an effect of a medical procedure on the patient based on the application of the model to the one or more heart rate variability features and the one or more clinical features; and
output the predicted effect of the medical procedure to the display device.

2. The computing system of claim 1, wherein the heart rate data comprises a plurality of time intervals corresponding to time periods between electrical signals recorded by the medical device, the electrical signals corresponding to depolarizations of a first chamber of a heart of the patient.

3. The computing system of claim 2, wherein the electrical signals comprise QRS complexes detected by the medical device, and wherein the time periods comprise times between R-waves of adjacent QRS complexes.

4. The computing system of claim 1, wherein the one or more heart rate variability features comprises:
an average value of the plurality of time intervals;
a mean square difference of adjacent time intervals of the plurality of time intervals;
a standard deviation of the plurality of time intervals; or
a percentage of the plurality of time intervals that satisfy a threshold time condition.

5. The computing system of claim 1, wherein the one or more clinical features of the patient comprises one or more of:
an age of the patient;
a presence of an illness in the patient; or
a length of a monitoring period of the patient prior to a past medical procedure.

6. The computing system of claim 5, wherein the illness comprises one or more of:
paroxysmal atrial fibrillation;
hypertension;
diabetes;
coronary artery disease;
lesions; or
stroke.

7. The computing system of claim 5, wherein the past medical procedure comprises a cardiac ablation procedure.

8. The computing system of claim 1, wherein the effect of a medical procedure on the patient comprises a recurrence of an atrial fibrillation (AF) episode experienced by the patient after performance of the medical procedure on the patient.

9. The computing system of claim 8, wherein the predicted effect of the medical procedure comprises a probability of the recurrence of the AF episode within a set period after the performance of the medical procedure on the patient.

10. The computing system of claim 1, wherein to apply the model, the processing circuitry is further configured to determine the model, and wherein to determine the model, the professing circuitry is further configured to:
select a first feature of the one or more heart rate variability features and the one or more clinical features for a prediction module;
determine a weight value for the first feature based on a plurality of classifier modules; and
generate a second prediction module comprising the first feature and the weight value.

11. The computing system of claim 10, wherein to select the first feature, the processing circuitry is configured to apply a sequential forward floating search (SFFS) to the one or more heart rate variability features and the one or more clinical features, and wherein to apply the SFFS, the processing circuitry is configured to:
select a first feature from the one or more heart rate variability features and the one or more clinical features;
apply a forward selection regression to the prediction module and the first feature; and
position the first feature within a position in the prediction module that maximizes the accuracy of the prediction module.

12. The computing system of claim 10, wherein to generate the second prediction module, the processing circuitry is further configured to:
select a second feature from a plurality of existing features in the prediction module;
apply a backward selection regression to the prediction module and the second feature; and
remove, based on a determination that removing the second feature increases the accuracy of the prediction module, the second feature from the prediction module.

13. The computing system of claim 10, wherein to determine the weight value, the processing circuitry is configured to:
apply a weighted voting system a to a plurality of weight vectors and a plurality of corresponding voting vectors, wherein each weight vector and the corresponding voting vector corresponds to one of the plurality of classifier modules; and
determine an accuracy of the prediction module associated with each of the plurality of weight vectors and the plurality of corresponding voting vector.

14. The computing system of claim 10, wherein the plurality of classifier modules comprises at least five classifier modules.

15. The computing system of claim 1, wherein the processing circuitry is configured to generate the model, and wherein to generate the model, the processing circuitry is configured to:
select a training set comprising a set of training instances, each training instance comprising an association between respective values for one or more of the one or more heart rate variability features or the one or more clinical features and a determined effect of the medical procedure; and
for each training instance in the training set, modify, based on particular values and a particular determined effect of the medical procedure, the model to change a likelihood predicted by the model for the particular predicted effect associated with the particular values in response to subsequent values for the one or more of the one or more heart rate variability features or the one or more clinical features applied to the model.

16. A method comprising:
collecting, by a computing system, heart rate data of a patient from a medical device of the patient;
determining, by the computing system, one or more heart rate variability features based on the heart rate data;
applying, by the computing system, a model to the one or more heart rate variability features and one or more clinical features of the patient;
predicting, by the computing system, an effect of a medical procedure on the patient based on the application of the model to the heart rate variability features and the one or more clinical features; and
outputting, by the computing system, the predicted effect of the medical procedure to a display device.

17. The method of claim 16, further comprising determining the model by at least:
selecting, by the computing system, a first feature of the one or more heart rate variability features and the one or more clinical features for a prediction module;
determining, by the computing system, a weight value for the first feature based on a plurality of classifier modules; and
generating, by the computing system, a second prediction module comprising the first feature and the weight value.

18. The method of claim 17,
wherein selecting the first feature comprises applying, by the computing system, sequential forward floating search (SFFS) to the one or more heart rate variability features and the one or more clinical features, wherein applying the SFFS comprises:
selecting, by the computing system, the first feature from the one or more heart rate variability features and the one or more clinical features;
applying, by the computing system, a forward selection regression to the prediction module and the first feature; and
positioning, by the computing system, the first feature within a position in the prediction module that maximizes the accuracy of the prediction module based on the forward selection regression.

19. The method of claim 17, wherein generating the second prediction module comprises:
selecting, by the computing system, a second feature from a plurality of existing features in the prediction module;
applying, by the computing system, a backward selection regression to the prediction module and the second feature; and
based on a determination, based on the backward selection regression, that removing the second feature increases accuracy of the prediction module, removing, by the computing system, the second feature from the prediction module.

20. A computer readable storage medium comprising instructions that when executed, cause processing circuitry within a device to:
collect heart rate data of a patient from a medical device of the patient;
determine one or more heart rate variability features based on the heart rate data;
apply a model to the one or more heart rate variability features and one or more clinical features of the patient;
predict an effect of a medical procedure on the patient based on the application of the model to the heart rate variability features and the one or more clinical features; and
cause a display device to output the predicted effect of the medical procedure.
